(19) United States
(12) Patent Application Publication
(10) Pub. No.: US 2002/0043656 A1
(43) Pub. Date: Apr. 18, 2002

(54) GREEN ORGANIC LUMINOPHOR

(76) Inventors: Victor M. Shershukov, Kharkov (UA); Valentina T. Skripkina, Kharkov (UA)

Correspondence Address: Patrick J. Coyne, Collier Shannon Scott, PLLC, Suite 400, 3050 K Street, N.W., Washington, DC, 20007, US

(21) Appl. No.: 09/827,184

(22) Filed: Apr. 6, 2001

(30) Foreign Application Priority Data
Apr. 6, 2000 (UA) 2000041949

(51) Int. Cl. C07D 471/06; G02B 5/23

(52) U.S. Cl. 252/586; 546/47

(57) ABSTRACT

The invention is an innovative, economical chemical compound 1-phenyl-5-(p-methoxyphenyl)-3-[1,8-naphthoylene-1′,2′-benzimidazol-4-yl]-2-pyrazol of the general formula 1:

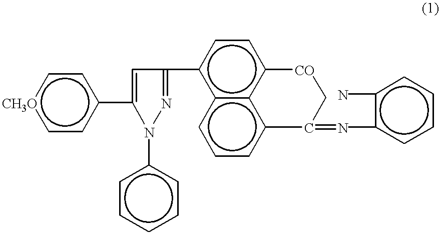

The compound is an efficient organic luminophor of green emission (λmax.lum>>510 nm) characterized by high quantum yield of photoluminescence: η=0.85 in toluene solution and η=0.78 in solid state (vacuum-deposited film) and large Stokes shift (FIGS. 1 and 2). The luminophor has increased solubility in organic solvents; its luminescence is increased over that of the prior art in, including, but not limited to, powder form, films and in solutions; and in exhibits increased photochemical and chemical stability.

### CROSS REFERENCE TO RELATED PATENT APPLICATIONS

This application is related to and claims priority on Ukrainian patent application serial number 2000041949, filed Apr. 6, 2000.

### FIELD OF THE INVENTION

This invention relates to a novel chemical compound 1phenyl-5-(p-methoxyphenyl)-3-[1,8-naphthoylene-1′,2′-benzimidazol-4-yl]-2-pyrazol of the general formula 1:

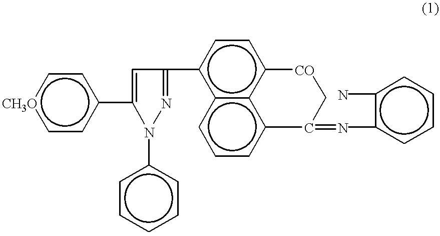

### BACKGROUND OF THE INVENTION

In the pyrazol derivative series, 2-methyl-5-methoxy-9-(4-dimethylaminophenyl)-2H-pyrazolo[4,3-b]quinoline is known having the formula:

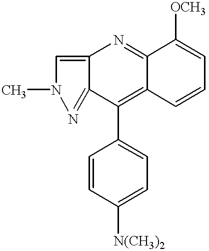

and exhibiting the properties of green emission organic luminophors (λmax.lum=510 nm, quantum yield η=0.8). The chemical structure and spectral properties of the compound according to an embodiment of the present invention are different from the prior art, as shown in the table, below.

The above-mentioned prior art luminophor was intended for use in luminescent flaw detection, polystyrene dyeing, and in gamma dosimeters (see Ref. 1, the contents of which are herein incorporated by reference). The production process of this luminophor is complex and includes several steps. Drawbacks of this luminophor include the multistage character of its production process. The synthesis includes three time-consuming and laborious stages using reagents which are hard to obtain, expensive, and damaging to the environment, such as phosphorus oxychloride, dimehylaniline, and copper metal. Complicated and laborious techniques, such as distillation and vapor distillation, are repeatedly performed. Moreover, the initial product for the first stage, 4-bromo-1-methylpyrazol-3-carboxylic acid, is not available commercially and must be prepared previously using a complex and environmentally damaging synthetic method. This process results in formation of many hazardous emissions and wastes. The luminophor is not produced industrially due to above-mentioned complexity and high price of its synthesis, and may be used for laboratory investigations only.

The compound's sensitivity to pH is also a drawback. This compound easily forms salts on the quinoline nitrogen atoms even in a weak acidic media which is typical of the ambient environment, and, in addition, loses its luminescent properties. This substantially restricts its practical application and requires a complete isolation against atmospheric factors, even under laboratory conditions.

A commercially produced green emission luminophor “Lumogen yellow-green”, namely, 1,8-naphthoylene-1′,2′-benzimidazol of formula:

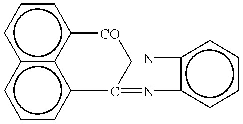

is also known. Its luminescence spectral maximum wavelength varies from 490 to 510 nm, depending on the luminophor concentration, the PL absolute quantum efficiency in toluene solution being η>>0.5 [3]. The luminophor is soluble in organic media and essentially water-insoluble. The luminescence is intense both in the powder form and in solutions. The compound is chemically stable against various reagents. Its preparation process is rather simple. The luminophor is produced by boiling of a mixture consisting of naphthalic anhydride and o-phenylene diamine in acetic acid and purified using chromatography. It is used to produce daylight fluorescent pigments, in luminescent flaw detection, plastics dyeing, sand labeling, etc. (See Ref. 2, 3, the contents of which are herein incorporated by reference).

Drawbacks of this compound substantially restricting its application include low quantum yield of fluorescence and insufficient emission intensity due to its low absorption. The extinction coefficient in the absorption maximum is 8000, while the compound according to an embodiment of the present invention may attains up to a 30,000 maximum absorption.

To overcome the problems of the prior art compounds, a compound according to an embodiment of the present invention has been developed to produce a high-efficiency luminescent liquid for flaw detection and to develop spectrum-shifting light guides with high conversion efficiency and high transparency to the proper emission as well as to produce dyes for plastics, novel radiation-resistant green emission organic luminophors exhibiting a high quantum yield and stable against chemical factors.

### REFERENCES

1. B. I. Stepanov, D. G. Pereyaslova, V. P. Perevalov, et al., USSR Authors Certificate No. 1314633, C07D 471/04; C09K 112/06, (1985) “2-methyl-5-methoxy-9-(4-dimethylaminophenyl)2H-pyrazolo[4,3-b]quinoline as a green emission organic luminophor”.

2. B. M. Krasovitskii, N. F. Levchenko, Yu. I. Makarenko, USSR Author's certificate No. 178821, Co7c; 12p3; 421/08, 1966; analog Brit. Pat. No. 1,081,677, int. Cl. C09k 1/02:C07d, 1967, “Lumogen yellow-green”.

3. B. M. Krasovitskii and B. M. Bolotin “Organic Luminescent Materials”, VCH Publishers, New York, 1988.

### OBJECTS OF THE INVENTION

It is therefore an object of the present invention to a novel, efficient, stable, and multiple-purpose luminophore with improved spectral properties.

It is another object of the present invention to provide a novel and efficient stable green luminophore.

It is a further object of the present invention to provide a novel compound characterized by the large Stokes shift and high quantum efficiency of photoluminescence.

It is still another object of the present invention to provide a luminophor that is soluble in organic solvents.

It is yet another object of the present invention to provide a luminophore with increased luminescence in powder form, films and in solutions.

It is still yet another object of the present invention to provide a luminophore that exhibits increased photochemical and chemical stability.

Additional objects and advantages of the invention are set forth, in part, in the description which follows and, in part, will be apparent to one of ordinary skill in the art from the description and/or from the practice of the invention.

### SUMMARY OF THE INVENTION

In response to the foregoing challenge, Applicants have developed an innovative, economical chemical compound 1-phenyl-5-(p-methoxyphenyl)-3-[1,8-naphthoylene-1′,2′-benzimidazol-4-yl]-2-pyrazol of general formula 1:

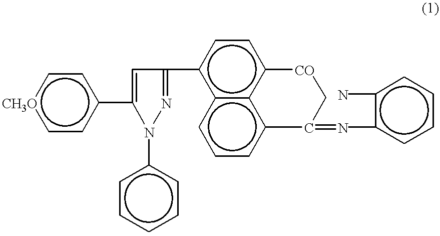

The proposed compound is an efficient organic luminophor of green emission (λmax.lum>>510 nm) characterized by high quantum yield of photoluminescence: η=0.85 in toluene solution and η=0.78 in solid state (vacuum-deposited film) and large Stokes shift as shown in FIGS. 1 and 2. The luminophor has increased solubility in organic solvents; its luminescence is increased over that of the prior art compounds in, including, but not limited to, powder form, films and in solutions; and it exhibits increased photochemical and chemical stability.

The improved properties enable the compound according to an embodiment of the present invention to be used successfully in, including, but not limited to, luminescent flaw detection, to offer luminescent colors of polymers, in scintillation engineering, as a spectrum shifter in polymer composites for spectrum-shifting light guides used for light collection in scintillation detectors (gamma spectrometers) as well as to convert the Cherenkov emission in deep-water neutrino detection, and for any other application requiring efficient green luminescence in solid state and solution.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention, and together with the detailed description serve to explain the principles of the present invention.

### BRIEF DESCRIPTION OF THE DRAWINGS

FIG. 1 is a graphical representation of the absorption and photoluminescence spectra in solution according to an embodiment of the present invention.

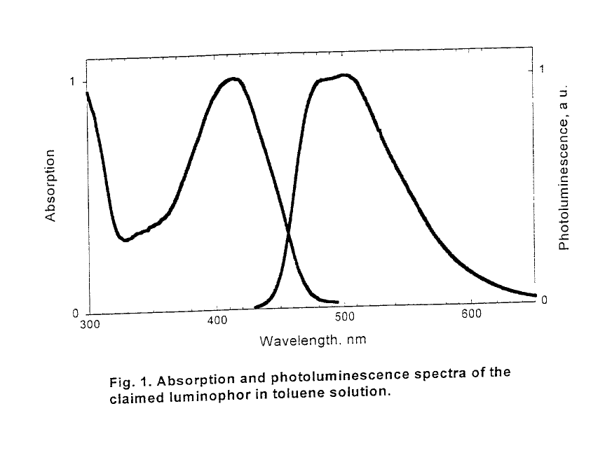

FIG. 2 is a graphical representation of the absorption and photoluminescence spectra in a film according to an embodiment of the present invention.

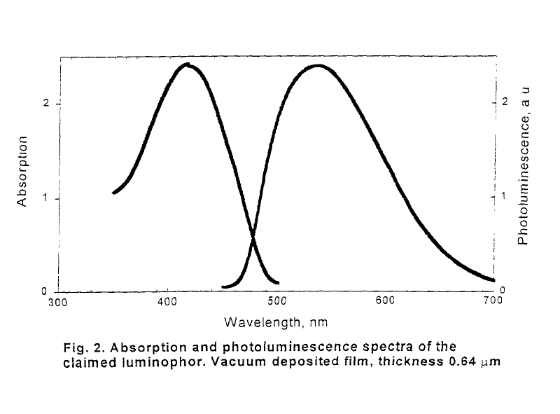

### DETAILED DESCRIPTION OF THE PREFERRED EMBODIMENTS

Reference will now be made in detail to a preferred embodiment of the present invention, a novel compound in the pyrazol derivative series having an enhanced emission intensity in the green spectral range, enhanced light stability and stability in aggressive media, and suitable for industrial production.

A preferred embodiment of the present invention is a novel chemical structure described by formula 1:

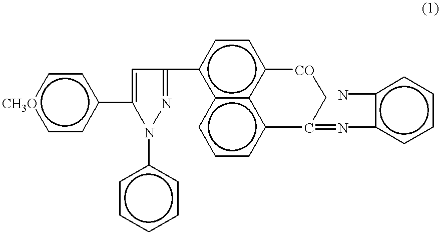

The compound of formula 1 is a modified pyrazol structure including pyrazol and naphthoylene-benzimidazol moieties. The structure modification provides an improvement in luminescent and spectrometric activity. The compound shows increased fluorescence intensity both in crystalline state as well in organic solvents and is an increased efficiency organic luminophor in the green spectral range; and it shows an enhanced quantum yield and an increased transparency to the proper emission.

An unanticipated effect is that the pyrazol molecular moiety exhibits essentially no luminescence while the naphthoylenebenzimidazol exhibits a relatively low quantum yield (η>>0.5); the combination of both moieties in one molecule results in a novel structure of efficient luminescence with quantum yield of approximately 0.82+0.05.

The above-mentioned properties of the compound according to a preferred embodiment of the present invention and its increased availability make it possible to use the novel compound widely and effectively for many applications (see Table).

A preferred embodiment of the chemical compound according to an embodiment of the present invention is produced by the oxidation of an appropriate pyrazoline derivative in acetic acid using potassium bichromate. The resulting solution poured into water, the precipitate formed is filtered, dried, and purified by chromatography.

The process is simple, environmentally friendly, and easy to realize both in the laboratory and in industrial applications.

Examples of the compound preparation:

### EXAMPLE 1

To the boiling solution of 1-phenyl-5-(p-methoxyphenyl)-3-[1,8-naphthoylene-1′,2′-benzimidazol-4-yl]-2-pyrazoline (20 g) in glacial acetic acid (400 ml), potassium bichromate (20 g) was added. The mixture was boiled during 1 hour, then cooled and poured into water (800 ml). The precipitated solid was filtered, water-washed until washings are colorless, dried, and chromatographed on aluminum oxide (solvent benzene). The compound was obtained as yellow crystals, water-insoluble, soluble in common organic solvents. M.p. 236-237° C. Yield: 16.9 g (85 %).

Calcul., %: C, 78.76; N, 10.8; H, 4.24.

Found, %: C, 78.46; N, 10.5; H, 4.2.

IR spectrum (tablets with KBr): v (cm⁻¹) 1695 (C═O); 1615 (benzene ring); 1595 (C═N). Absorption (in toluene): λ=412 nm. Luminescence (in toluene): λ=495 nm.

### EXAMPLE 2

The compound was prepared as is described in Example 1 except for the mixture was boiled for 2 hours. The compound obtained has m.p. 236-237° C. Yield: 15.7 g. (79%)

IR, absorption and luminescence spectra were identical to those described in Example 1.

### EXAMPLE 3

The compound was prepared as is described in Example 1 except for 25 g. of potassium bichromate were used. The compound obtained has m.p. 236-237° C. Yield: 15.5 g. (78%).

IR, absorption and luminescence spectra were identical to those described in Example 1.

1-phenyl-5-(p-methoxyphenyl)-3-[1,8-naphthoylene-1′,2′-benzimidazol-4-yl]-2-pyrazol was assayed as organic luminophor. Fluorescence spectra in toluene were measured and absolute fluorescence quantum yields in crystals were determined.

The fluorescence spectra were measured using a setup including a 3MR-3 mirror monochromator, a FEU-1 8 photomultiplier, an M-95 microampermeter. A SVDT-95 lamp was used as the excitation source, the 365 nm exciting line was singled out of its spectrum.

The absolute quantum yield was determined using the equal absorption technique (A. S. Cherkasov, Z. Fiz. Khim. 29, 2209, 1995).

The compound structure was confirmed by electron absorption spectra and IR ones.

The measured data for the claimed compound in comparison with the structure analog and the emission range one are presented in the Table below.

| | TABLE | |
|---|---|---|
| | Luminescence in | |
| | toluene | |
| Compound | λₘₐₓ, nm | η |
| The compound according to an embodiment of the | 510 | 0.85 |
| present invention: | | |
| 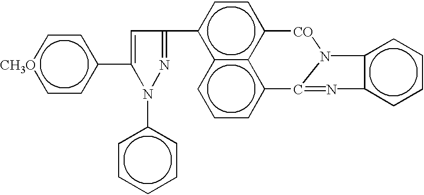 | | |
| Prior art structure analog: | 510 | 0.8 |
| 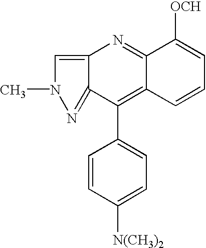 | | |
| Prior art emission range analog: | 490 | 0.5 |
| 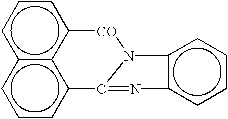 | | |

It follows from the description and Table, the compound according to an embodiment of the present invention exceeds the structure analog (prior art) in the emission intensity; the emission range analog is exceeded in the emission intensity by almost two-fold.

The advantage of the compound according to an embodiment of the present invention as compared to the prior art includes enhanced stability against aggressive media as well as in that the production method of an embodiment of the present invention is simplified and significantly shortens the process duration, does not require the use of environmentally unfriendly and scarce materials and is easy to realize on an industrial scale.

It will be apparent to those skilled in the art that various modifications and variations can be made in the construction, configuration, and/or operation of the present invention without departing from the scope or spirit of the invention. For example, in the embodiments mentioned above, various changes may be made to the synthesis without departing from the scope and spirit of the invention. Further, it may be appropriate to make additional modifications or changes to the purification scheme without departing from the scope of the invention. Thus, it is intended that the present invention cover the modifications and variations of the invention provided they come within the scope of the appended claims and their equivalents.

We claim:

1. 1-Phenyl-5-(p-methoxyphenyl)-3-[1,8-naphthoylene-1′,2′-benzimidazol-4-yl)-2-pyrazol of the formula: 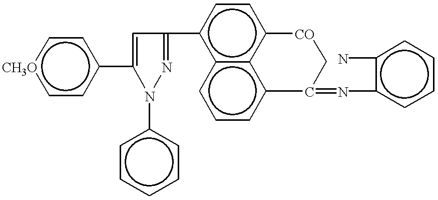
as a green emission organic luminophor.
